(19) United States
(12) Patent Application Publication
(10) Pub. No.: US 2025/0235219 A1
(43) Pub. Date: Jul. 24, 2025

(54) THROMBUS REMOVAL SYSTEMS AND ASSOCIATED METHODS

(71) Applicant: Shifamed Holdings, LLC, Campbell, CA (US)

(72) Inventors: Paul GUNNING, Montara, CA (US); Amr SALAHIEH, Saratoga, CA (US); Aadel AL-JADDA, San Carlos, CA (US)

(21) Appl. No.: 18/847,594

(22) PCT Filed: Mar. 15, 2023
(86) PCT No.: PCT/US2023/064486

§ 371 Date: Sep. 16, 2024

Related U.S. Application Data
(60) Provisional application No. 63/269,380, filed on Mar. 15, 2022.

(51) Int. Cl. A61B 17/22; A61B 17/00

(52) U.S. Cl. CPC A61B 17/22; A61B 2017/00292; A61B 2017/00561; A61B 2017/00778; A61B 2017/00867; A61B 2017/22079

(57) ABSTRACT

The present technology relates to systems and methods for removing a thrombus from a blood vessel of a patient. In some embodiments, the present technology is directed to systems including an elongated catheter having a distal portion configured to be positioned within the blood vessel of the patient, a proximal portion configured to be external to the patient, and a lumen extending therebetween. The system can also include a fluid delivery mechanism coupled with a fluid lumen and configured to apply fluid to at least partially fragment the thrombus.

### CROSS REFERENCE TO RELATED APPLICATIONS

This application claims the benefit of priority to U.S. Application No. 63/269,380, filed Mar. 15, 2022, which is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present technology generally relates to medical devices and, in particular, to systems including aspiration and fluid delivery mechanisms and associated methods for removing a thrombus from a mammalian blood vessel.

### BACKGROUND

Thrombotic material may lead to a blockage in fluid flow within the vasculature of a mammal. Such blockages may occur in varied regions within the body, such as within the pulmonary system, peripheral vasculature, deep vasculature, or brain. Pulmonary embolisms typically arise when a thrombus originating from another part of the body (e.g., a vein in the pelvis or leg) becomes dislodged and travels to the lungs. Anticoagulation therapy is the current standard of care for treating pulmonary embolisms, but may not be effective in some patients. Additionally, conventional devices for removing thrombotic material may not be capable of navigating the vascular anatomy of the lungs, may not be effective in removing thrombotic material, and/or may lack the ability to provide sensor data or other feedback to the clinician during the thrombectomy procedure.

### SUMMARY OF THE DISCLOSURE

A thrombus removal device, comprising an elongate catheter having an aspiration lumen configured to be coupled to an aspiration source, an expandable funnel coupled to the aspiration lumen and the elongate catheter, the expandable funnel including a proximal flared portion near the aspiration lumen and a distal flared portion near a distal end of the expandable funnel, one or more fluid ports disposed near or within the expandable funnel defining a fluid region.

In some aspects, the device can further include an aspiration source and/or a fluid source.

In some aspects, a first diameter of the expandable funnel at the proximal flared portion is within 10% of a second diameter of the expandable funnel at the distal flared portion.

In one aspect, a first diameter of the expandable funnel at the proximal flared portion is within 20% of a second diameter of the expandable funnel at the distal flared portion.

In other aspects, the proximal flared portion is configured to facilitate advancement of clots into the fluid region.

In some aspects, the distal end of the expandable funnel is directed radially outwards.

In some aspects, the distal end of the expandable funnel curves proximally back towards the distal flare.

In one aspect, the distal end is not bonded to the distal flare.

In some aspects, the distal end is bonded to the distal flare.

In some aspects, the expandable funnel further comprises a funnel frame.

In one aspect, the funnel frame comprises a shape memory material.

In some aspects, the funnel frame comprises a plurality of axially extending spines and one or more layers of radially and/or circumferentially extending struts between adjacent spines.

In additional aspects, the proximal flare is formed as a result of a bend in each of the plurality of axially extending spines.

In one aspect, the distal flare is formed as a result of a bend in each of the plurality of radially and/or circumferentially extending struts.

In one aspect, the device further includes at least one hinge in each of the plurality of radially and/or circumferentially extending struts.

In some aspects, a first hinge is disposed at a distal most portion of each of the plurality of radially and/or circumferentially extending struts.

In other aspects, [a second hinge is disposed at a junction between one of the plurality of radially and/or circumferentially extending struts and one of the plurality of axially extending spines.

In some aspects, the at least one hinge is configured to reducing a sheathing force of the expandable funnel.

In one aspect, the at least one hinge is integral to the plurality of radially and/or circumferentially extending struts.

In one aspect, the plurality of axially extending spines are stiffer than the plurality of radially and/or circumferentially extending struts.

In other aspects, the plurality of axially extending spines have a thickness greater than that of the plurality of radially and/or circumferentially extending struts.

In some aspects, the funnel frame comprises two layers of radially and/or circumferentially extending struts.

In one aspect, the plurality of axially extending spines and radially and/or circumferentially extending struts collectively form a plurality of petals in each of the one or more layers.

In other aspects, petals in a proximal layer of the funnel frame are smaller than petals in a distal layer of the funnel frame.

In some aspects, the device includes at least six petals in each of the one or more layers.

In one aspect, the device includes a compliant material attached to the funnel frame.

In another aspect, a distal-most portion of the expandable funnel comprises only compliant material with no funnel frame.

In one aspect, a distal-most extent of the funnel frame is proximal to a distal-most portion of the expandable funnel.

A catheter device is provided, comprising an elongate catheter having lumen therethrough, an expandable funnel coupled to the elongate catheter at a distal end thereof, the expandable funnel including a funnel frame comprising a plurality of axially extending spines, one or more layers of radially and/or circumferentially extending struts between adjacent spines, and one or more hinges disposed on or in the plurality of radially extending struts, the one or more hinges adapted to flex circumferentially and/or radially to modify a geometry of the expandable funnel.

In some aspects, the expandable funnel further comprises a proximal flared portion near the elongate catheter and a distal flared portion near a distal end of the expandable funnel.

In another aspect, a first diameter of the expandable funnel at the proximal flared portion is within 10% of a second diameter of the expandable funnel at the distal flared portion.

In one aspect, a first diameter of the expandable funnel at the proximal flared portion is within 20% of a second diameter of the expandable funnel at the distal flared portion.

In another aspect, a plurality of jet ports are disposed near or within the expandable funnel, and a fluid source coupled to the plurality of jet ports and configured to produce a plurality of fluid steams within or near the expandable funnel.

In one aspect, a distal end of the expandable funnel is directed radially outwards.

In one aspect, a distal end of the expandable funnel curves proximally back towards the expandable funnel.

In another aspect, the distal end is not bonded to the distal flare.

In some aspects, the distal end is bonded to the distal flare.

In some aspects, the funnel frame comprises a shape memory material.

In one aspect, the proximal flare is formed as a result of a bend in each of the plurality of axially extending spines.

In some aspects, the distal flare is formed as a result of a bend in each of the plurality of radially extending struts.

In one aspect, a first hinge is disposed at a distal most portion of each of the plurality of radially extending struts.

In other aspects, a second hinge is disposed at a junction between one of the plurality of radially extending struts and one of the plurality of axially extending spines.

In some aspects, the one or more hinges is integral to the plurality of radially extending struts.

In one aspect, the plurality of axially extending spines are stiffer than the plurality of radially extending struts.

In some aspects, the plurality of axially extending spines have a thickness greater than that of the plurality of radially extending struts.

In some aspects, the funnel frame comprises two layers of radially extending struts.

In one aspect, the plurality of axially extending spines and radially extending struts collectively form a plurality of petals in each of the one or more layers.

In some aspects, petals in a proximal layer of the funnel frame are smaller than petals in a distal layer of the funnel frame.

In some aspects, there are at least six petals in each of the one or more layers.

In another aspect, a compliant material is attached to the funnel frame.

In one aspect, a distal-most portion of the expandable funnel comprises only compliant material with no funnel frame.

In another aspect, the compliant material comprises a first compliant material layer on an interior of the expandable funnel and a second compliant material layer on an exterior of the expandable funnel.

In other aspects, the first compliant material layer is lubricious.

In some aspects, the first compliant material layer includes PTFE or ePTFE.

In another aspect, a delivery sheath is configured to move axially over the elongate catheter and the expandable funnel.

In one aspect, advancing the delivery sheath over the funnel causes the radially extending struts to bend or fold at the plurality of hinges.

In another aspect, advancing the delivery sheath over the funnel causes a distal end of the funnel frame to move inwards without flaring radially outwards.

A thrombus removal device is provided, comprising an elongate catheter having an aspiration lumen, an aspiration source coupled to the aspiration lumen, and an expandable funnel coupled to the aspiration lumen and the elongate catheter, the expandable funnel comprising a distal end thereof that is flared radially outward with respect to proximal portions thereof.

In some aspects, the expandable funnel comprises compound curvature along its axial dimension.

In another aspect, the compound curvature comprises an inversion in curvature.

In some aspects, the distal end comprises the inversion in curvature.

In one aspect, the expandable funnel further comprises a frame.

In some aspects, a distal extent of the frame is proximal to the distal end of the expandable funnel.

In one aspect, the distal end of the expandable funnel is relatively more compliant than a remainder of the expandable funnel.

A thrombus removal device is provided, comprising an elongate catheter having an aspiration lumen, an aspiration source coupled to the aspiration lumen, and an expandable funnel coupled to the aspiration lumen and the elongate catheter, the expandable funnel comprising an aspiration element disposed at a distal portion thereof, the aspiration element adapted to enhance an aspiration force in a region of the expandable funnel adjacent thereto.

In some aspects, the aspiration element comprises a skirt (or apron) is attached to the expandable funnel at one end, wherein the skirt comprises a surface configured to move freely.

In some aspects, the surface is adapted to be moved proximally.

In other aspects, the surface is adapted to interface with a clot.

In one aspect, the aspiration element comprises a protrusion (rib) that extends radially inward from, and at least partially about, an inner perimeter of the expandable funnel.

In some aspects, the protrusion extends fully about the inner perimeter of the expandable funnel.

In one aspect, the protrusion is disposed at an axial position of the expandable funnel.

A method for removing thrombus from a blood vessel of a patient with a thrombus removal device is provided, the method comprising: introducing a distal portion of an elongate catheter having an expandable funnel in a blood vessel to a target location near the thrombus, everting a distal end of the expandable funnel, operating an aspiration source of the elongate catheter, and removing the thrombus from the patient with the aspiration source through the thrombus removal device.

In some aspects, the method includes macerating the thrombus within the funnel and/or the distal portion of the elongate catheter.

In one aspect, the everting comprising approximating the thrombus with actuation elements to enhance the macerating.

In another aspect, macerating comprises irrigating the thrombus with one or more fluid streams.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1-1L illustrate various views of a portion of a thrombus removal system including a distal portion of an elongated catheter configured in accordance with an embodiment of the present technology.

FIGS. 2A-2E illustrate plan views of various configurations of irrigation ports and fluid streams of a thrombus removal system according to embodiments of the present technology.

FIGS. 3A-3H illustrate an elevation view of various configurations of irrigation ports of a thrombus removal system according to embodiments of the present technology.

FIGS. 4A-4H illustrate an elevation view of various configurations of irrigation ports and fluid streams of a thrombus removal system according to embodiments of the present technology.

FIGS. 5A-5G illustrate various configurations of irrigation ports of a thrombus removal system according to embodiments of the present technology.

FIGS. 6A-6C illustrate various embodiments of a thrombus removal system including a saline source, an aspiration system, and one or more controls for controlling irrigation and/or aspiration of the system.

FIGS. 7A-7E show a funnel portion of a thrombectomy catheter can include a shape that promotes clot capture during aspiration.

FIGS. 8A-8C illustrate various embodiments of a funnel distal tip.

FIG. 9A-9C shows an example of a funnel transitioning to an everted configuration.

FIGS. 10A-10B illustrate two embodiments of a funnel and funnel frame.

FIGS. 10C-10F illustrate sheathing of the funnels of FIGS. 10A-10B.

FIG. 11 is a chart showing the sheathing forces required for the funnels of FIGS. 10A-10B.

FIGS. 12A-12D illustrate aspiration elements adapted to enhance aspiration forces within the thrombectomy catheter.

### DETAILED DESCRIPTION

This application is related to disclosure in International Application No. PCT/US2021/020915, filed Mar. 4, 2021, the disclosure of which is incorporated by reference herein for all purposes.

The present technology is generally directed to thrombus removal systems and associated methods. A system configured in accordance with an embodiment of the present technology can include, for example, an elongated catheter having a distal portion configured to be positioned within a blood vessel of the patient, a proximal portion configured to be external to the patient, a fluid delivery mechanism configured to fragment the thrombus with pressurized fluid, an aspiration mechanism configured to aspirate the fragments of the thrombus, and one or more lumens extending at least partially from the proximal portion to the distal portion.

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples but are not described in detail with respect to the figures.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “generally,” “approximately,” and “about” are used herein to mean the stated value plus or minus 10%.

Although some embodiments herein are described in terms of thrombus removal, it will be appreciated that the present technology can be used and/or modified to remove other types of emboli that may occlude a blood vessel, such as fat, tissue, or a foreign substance. Additionally, although some embodiments herein are described in the context of thrombus removal from a pulmonary artery (e.g., pulmonary embolectomy), the technology may be applied to removal of thrombi and/or emboli from other portions of the vasculature (e.g., in neurovascular, coronary, or peripheral applications). Moreover, although some embodiments are discussed in terms of maceration of a thrombus with a fluid, the present technology can be adapted for use with other techniques for breaking up a thrombus into smaller fragments or particles (e.g., ultrasonic, mechanical, enzymatic, etc.).

The headings provided herein are for convenience only and do not interpret the scope or meaning of the claimed present technology.

### Systems for Thrombus Removal

As provided above, the present technology is generally directed to thrombus removal systems. Such systems include an elongated catheter having a distal portion positionable within a blood vessel of the patient (e.g., an artery or vein), a proximal portion positionable outside the patient's body, a fluid delivery mechanism configured to fragment the thrombus with pressurized fluid, an aspiration mechanism configured to aspirate the fragments of the thrombus, and one or more lumens extending at least partially from the proximal portion to the distal portion. In some embodiments, the systems herein are configured to engage a thrombus in a patient's blood vessel, break the thrombus into small fragments, and aspirate the fragments out of the patient's body. The pressurized fluid streams (e.g., jets) function to cut or macerate thrombus, before, during, and/or after at least a portion of the thrombus has entered the aspiration lumen or a funnel of the system. Fragmentation helps to prevent clogging of the aspiration lumen and allows the thrombus removal system to macerate large, firm clot that otherwise could not be aspirated. As used herein, “thrombus” and “embolism” are used somewhat interchangeably in various respects. It should be appreciated that while the description may refer to removal of “thrombus,” this should be understood to encompass removal of thrombus fragments and other emboli as provided herein.

According to embodiments of the present technology, a fluid delivery mechanism can provide a plurality of fluid streams (e.g., jets) to fluid apertures of the thrombus removal system for macerating, cutting, fragmenting, pulverizing and/or urging thrombus to be removed from a proximal portion of the thrombus removal system. The thrombus removal system can include an aspiration lumen extending at least partially from the proximal portion to the distal portion of the thrombus removal system that is adapted for fluid communication with an aspiration pump (e.g., vacuum source). In operation, the aspiration pump may generate a volume of lower pressure within the aspiration lumen near the proximal portion of the thrombus removal system, urging aspiration of thrombus from the distal portion.

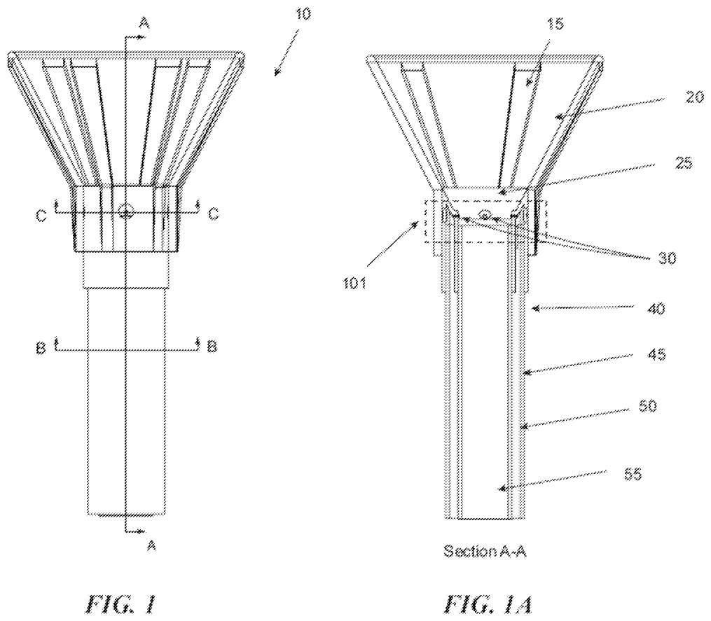

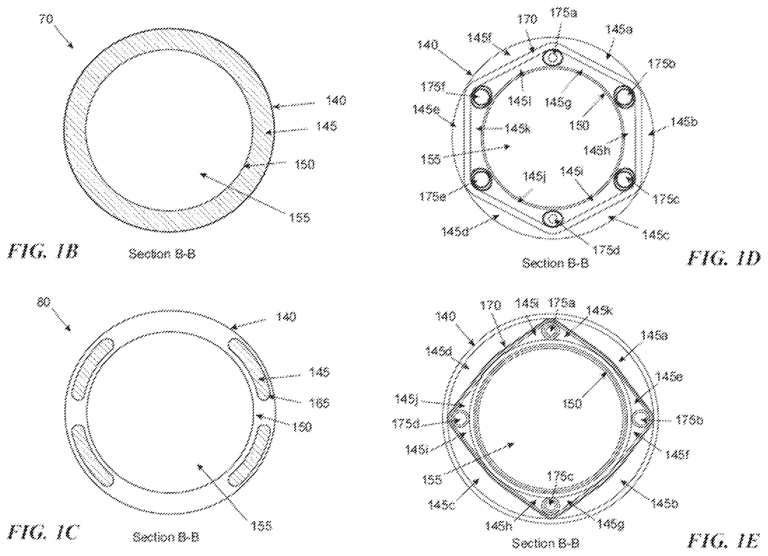

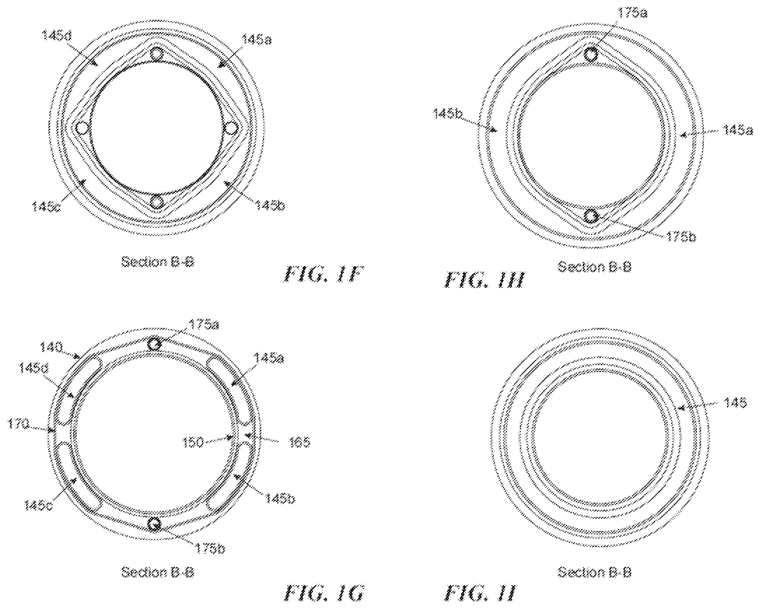

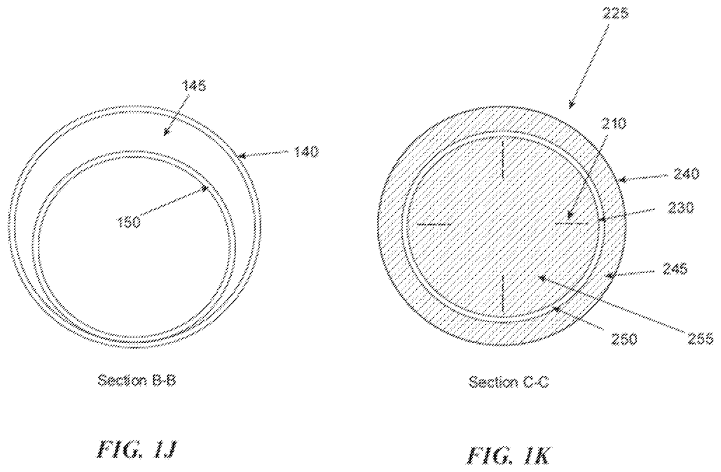

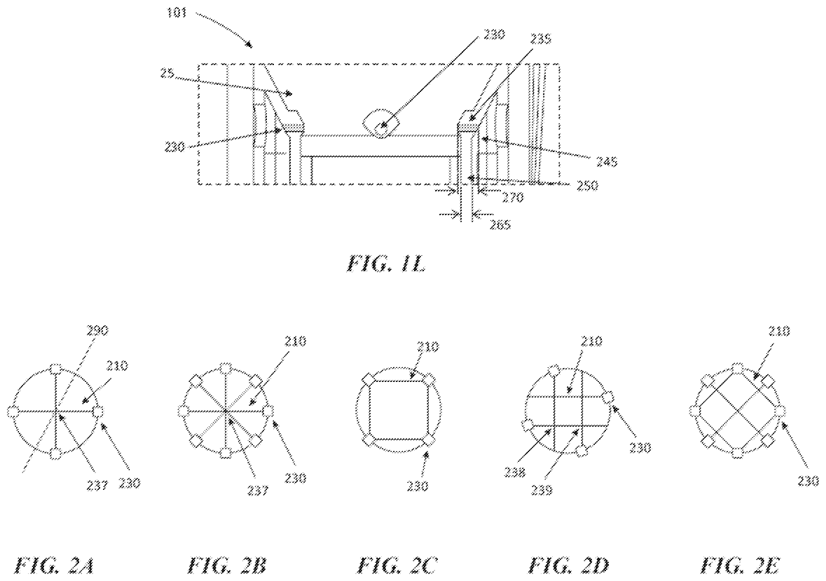

FIG. 1 illustrates a distal portion 10 of a thrombus removal system according to an embodiment of the present technology. FIG. 1A Section A-A illustrates an elevation sectional view of the distal portion. The example section A-A in FIG. 1A depicts a funnel 20 that is positioned at the distal end of the distal portion 10, the funnel adapted to engage with thrombus and/or a tissue (e.g., vessel) wall to aid in thrombus fragmentation and/or removal. The funnel can be formed according to any of the constructions described herein. The example section A-A in FIG. 1A depicts a double walled thrombus removal device construction having an outer wall/tube 40 and an inner wall/tube 50. An aspiration lumen 55 is formed by the inner wall 50 and is centrally located. A generally annular volume forms at least one fluid lumen 45 between the outer wall 40 and the inner wall 50. The fluid lumen 45 is adapted for fluid communication with the fluid delivery mechanism. One or more apertures (e.g., nozzles, orifices, or ports) 30 are positioned in the thrombus removal system to be in fluid communication with the fluid lumen 45 and an irrigation manifold 25. In operation, the ports 30 are adapted to direct (e.g., pressurized) fluid toward thrombus that is engaged with the distal portion 10 of the thrombus removal system.

In various embodiments, the system can have an average flow velocity within the fluid lumen of at least 20 m/s to achieve consistent and successful aspiration of clots. In some embodiments, the fluid source itself can be delivered in a pulsed sequence or a preprogrammed sequence that includes some combination of pulsatile flow and constant flow to deliver fluid to the jets. In these embodiments, while the average pulsed fluid velocity may be at least 20 m/s, the peak fluid velocity may be up to 30 m/s or more during the pulsing of the fluid source. In some embodiments, the jets or apertures are no smaller than 0.0100″ or even as small as 0.008″ to avoid undesirable spraying of fluid. In some embodiments, the system can have a minimum vacuum or aspiration pressure of 15 inHg, to achieve the desired performance necessary to remove target clots after they have been macerated or broken up with the jets described above.

The thrombus removal system can be sized and configured to access and remove thrombi in various locations or vessels within a patient's body. It should be understood that while the dimensions of the system may vary depending on the target location, generally the same features and components described herein will be implemented in the thrombus removal system regardless of the application. For example, a thrombus removal system configured to remove pulmonary embolism (PE) from a patient may have an outer wall/tube with a size of approximately 11-13 Fr, or preferably 12 Fr, and an inner wall/tube with a size of 7-9 Fr, or preferably 8 Fr. A deep vein thrombosis (DVT) device, on the other hand, may have an outer wall/tube with a size of approximately 9-11 Fr, or preferably 10 Fr, and an inner wall/tube with a size of 6-9 Fr, or preferably 7.5 Fr. Applications are further provided for ischemic stroke and peripheral embolism applications.

Section B-B of FIG. 1B illustrates in plan view a portion of the thrombus removal system that is proximal to the funnel and irrigation manifold. Section B-B depicts an outer wall 140, an inner wall 150, an aspiration lumen 155 and a fluid lumen 145. In some embodiments, in cross-section the aspiration lumen 155 is generally circular and the fluid lumen 145 is generally annular in shape (e.g., cross-section 70). It will be appreciated that alternative constructions and/or arrangements of the inner wall 150 and the outer wall 140 produce variations in cross-sectional shape of the aspiration and fluid lumens 155 and 145. For example, the inner wall 150 can be shaped to form an aspiration lumen 155 that, in cross-section, is generally oval, circular, rectilinear, square, pentagonal, or hexagonal. The inner and outer walls 150 and 140 can be shaped and arranged to form a fluid lumen 145 that, in cross-section, is generally crescent-shaped, diamond shaped, or irregularly shaped. For example, referring to FIG. 1C Section B-B, the region between the inner wall 150 and the outer wall 140 can include one or more wall structures 165 that form respective fluid lumens 145 (e.g., as in cross-section 80). The wall structures 165 can be formed by lamination between the outer and inner walls 140 and 150, or by a multi-lumen extrusion that forms a plurality of the wall structures.

Section B-B of FIGS. 1D-1H illustrate additional examples of a portion of the thrombus removal system that is proximal to the funnel and irrigation manifold. Similar to the embodiments described above, the portion in these examples can include an outer wall 140, an inner wall 150, and an aspiration lumen 155. Additionally, the illustrated portion of the thrombus removal system can include a middle wall 170 disposed between the outer wall 140 and the inner wall 150. The middle wall 170 enables further segmentation of the annular space between the inner wall and outer wall into a plurality of distinct fluid lumens and/or auxiliary lumens. For example, referring to FIG. 1D, the middle wall can be generally hexagon shaped, and the annular space can include a plurality of fluid lumens 145a-14l and a plurality of auxiliary lumens 175a-175f. As shown in FIG. 1D, the fluid lumens can be formed by some combination of the outer wall 140 and the middle wall 170, or between the middle wall 170, the inner wall 150, and two of the auxiliary lumens. For example, fluid lumen 145a is formed in the space between outer wall 140 and middle wall 170. However, fluid lumen 145g is formed in the space between middle wall 170, inner wall 150, auxiliary lumen 175a, and auxiliary lumen 175b. Generally, the fluid lumens are configured to carry a flow of fluid such as saline from a saline source of the system to one or more ports/apertures/orifices of the system. The auxiliary lumens can be configured for a number of functions. In some embodiments, the auxiliary lumens can be coupled to the fluid/saline source and to the apertures to be used as additional fluid lumens. In other embodiments, the auxiliary lumens can be configured as steering ports and can include a guide wire or steering wire within the lumen for steering of the thrombus removal system. Additionally, in other embodiments, the auxiliary lumens can be configured to carry electrical, mechanical, or fluid connections to one or more sensors. For example, the system may include one or more electrical, optical, or fluid based sensors disposed along any length of the system. The sensors can be used during therapy to provide feedback for the system (e.g., sensors can be used to detect clogs to initiate a clog removal protocol, or to determine the proper therapy mode based on sensor feedback such as jet pulse sequences, aspiration sequences, etc.). The auxiliary ports can therefore be used to connect to the sensors, e.g., by electrical connection, optical connection, mechanical/wire connection, and/or fluid connection. It is also contemplated that the fluid and auxiliary lumens can be configured to carry and deliver other fluids, such as thrombolytics or radio-opaque contrast injects to the target tissue site during treatment.

It should be understood that in some embodiments, all the fluid lumens are fluidly connected to all of the jets or apertures of the thrombus removal device. Therefore, when a flow of fluid is delivered from the fluid lumen(s) to the jets, all jets are activated with a jet of fluid at once. However, it should also be understood that in some embodiments, the fluid lumens are separate or distinct, and these distinct fluid lumens may be fluidly coupled to one or more jets but not to all jets of the device. In these embodiments, a subset of the jets can be controlled by delivering fluid only to the fluid lumens that are coupled to that subset of jets. This enables additional functionality in the device, in which specific jets can be activated in a user defined or predetermined order.

Section B-B of FIG. 1E illustrates another embodiment of the portion of the thrombus removal system that is proximal to the funnel and irrigation manifold. Similar to the embodiment of FIG. 1D, this embodiment also includes a middle wall 170. However, the middle wall in this example is generally square shaped, facilitating the formation of fluid lumens 145a-145k and auxiliary lumens 175a-175d. The example illustrated in section B-B of FIG. 1F is similar to that of the embodiment of FIG. 1E, however this embodiment includes only fluid lumens 145a-145d. The fluid lumens 145e-145k from the embodiment of FIG. 1E are not used as fluid lumens in this embodiment. They can be, for example, empty lumens, vacuum, filled with an insulative material, and/or filled with a radio-opaque material or any other material that may help visualize the thrombus removal system during therapy. The embodiment 1F includes the same four auxiliary reports as illustrated and described in the embodiment of FIG. 1E.

Section B-B of FIG. 1G illustrates another example of a portion of the thrombus removal system that is proximal to the funnel and irrigation manifold. Similar to the embodiments described above, the illustrated portion of the thrombus removal system can include a middle wall 170 disposed between the outer wall 140 and the inner wall 150. However, this embodiment includes four distinct fluid lumens 145a-145d formed by wall structures 165. As with the embodiment of FIG. 1C, the wall structures 165 can be formed by lamination between the outer and inner walls 140 and 150, or by a multi-lumen extrusion that forms a plurality of the wall structures. As shown, this embodiment can include a pair of auxiliary lumens 175a and 175b, which can be used, for example, for steering or for sensor connections as described above.

Section B-B of FIG. 1H is another similar embodiment in which the middle wall and outer wall can be used to form fluid lumens 145a and 145b. Auxiliary lumens 175a and 175b can be formed in the space between the middle wall and the inner wall. It should be understood that the middle wall can contact the outer wall to create independent fluid lumens 145a and 145b. However, in other embodiments, it should be understood that the middle wall may not contact the outer wall, which would facilitate a single annular fluid lumen, such as is shown by fluid lumen 145 in Section B-B of FIG. 1I. In another embodiment, as shown in Section B-B of FIG. 1J, the inner wall 150 and the outer wall 140 may not be concentric, which facilitates formation of an annular space and/or fluid lumen 145 that is thicker or wider on one side of the device relative to the other side. As shown in FIG. 1J, a distance between the outer wall 140 and inner wall at the top (e.g., 12 o'clock) portion of the device is larger than a distance between the outer wall and inner wall at the bottom (e.g., 6 o'clock) portion of the device.

Section C-C of FIG. 1K illustrates in plain view a portion of the thrombus removal system comprising an irrigation manifold 225. Section C-C depicts an outer wall 240, an inner wall 250, a fluid lumen 245, an aspiration lumen 255, and ports 230 for directing respective fluid streams 210.

Detail View 101 of FIG. 1L illustrates a section view in elevation of a portion of the irrigation manifold 25 that includes a plurality of ports 230 that are formed within an inner wall 250. In some embodiments, a thickness of one or more walls of the thrombus removal system may be varied along its axial length and/or its circumference. As shown in Detail View 101, inner wall 250 has a first thickness 265 in a region 250 that is proximal to the irrigation manifold 25, and a second thickness 270 in a region 235 that includes the ports 230. In some embodiments, the second thickness 270 is greater than the first thickness 265. The first thickness 265 can correspond to a general wall thickness of the inner wall 50 and/or of the outer wall 40, which can be from about 0.10 mm to about 0.60 mm, or any value within the aforementioned range. The second thickness 270 can be from about 0.20 mm to about 0.70 mm, from about 0.70 mm to about 0.90 mm, or from about 0.90 mm to about 1.20 mm. The second thickness 270 can be any value within the aforementioned range. The dimension of the second thickness 270 can be selected to provide a fluid path through the ports 230 that produces a generally laminar flow for a fluid stream that is directed therethrough, when the fluid delivery mechanism supplies fluid via the fluid lumen 245 at a typical operating pressure. Such operating pressure can be from about 10 psi to about 60 psi, from about 60 psi to about 100 psi, or from about 100 psi to about 150 psi. The operating pressure of the fluid delivery mechanism can be any value within the aforementioned range of values. In some embodiments, the fluid delivery mechanism is operated in a high pressure mode, having a pressure from about 150 psi to about 250 psi, from about 250 psi to about 350 psi, from about 350 psi to about 425 psi, or from about 425 psi to about 500 psi. The operating pressure of the fluid delivery mechanism in the high pressure mode can be any value within the aforementioned range of values. Generally, the length of the aperture or hole through the walls that is used to form the ports 230 needs to have a sufficient diameter to prevent formation of a spray or mist and a sufficient length to create laminar flow as the fluid exits the ports. Instead, a focused jet or stream is desired. Given the parameters described above, the length of the apertures through the walls that are used to form the ports should be at least 0.25 mm in length, optionally up to 0.4 mm or up to 1 mm or greater in length. Any lengths shorter than that may undesirably lead to mist or spray ejection from the ports, which will not effectively break up or macerate target clots.

In some embodiments, a profile (cross-sectional dimension) of a port 230 varies along its length (e.g., is non-cylindrical). A variation in the cross-sectional dimension of the port may alter and/or adjust a characteristic of fluid flow along the port 230. For example, a reduction in cross-sectional dimension may accelerate a flow of fluid through the port 230 (for a given volume of fluid). In some embodiments, a port 230 may be conical along its length (e.g., tapered), such that its smallest dimension is positioned at the distal end of the port 230, where distal is with respect to a direction of fluid flow.

In some embodiments, the port 230 is formed to direct the fluid flow along a selected path. FIGS. 2A-2E illustrate various embodiments of arrangements of ports 230 for directing respective fluid streams 210. In some embodiments, such as those shown in FIGS. 2A and 2B, at least two ports 230 are arranged to produce (e.g., respective) fluid streams 210 that intersect at an intersection region 237 of the thrombus removal system. An intersection region 237 can be a region of increased fluid momentum and/or energy transfer, which increase is with respect to individual fluid streams that are not directed to combine at the intersection. The increased fluid momentum and/or energy transfer at an intersection may advantageously fragment thrombus more efficiently and/or quickly. In some embodiments, an intersection region can be formed from at least 2, at least 3, at least 4, at least 5, at least 6, at least 7, at least 8, at least 9, or at least 10 fluid streams 210. An intersection region can be generally near a central axis 290 of the thrombus removal system (e.g., 237), or away from the central axis (e.g., 238 and 239 in the embodiment of FIG. 2D). In some embodiments, at least two intersection regions (e.g., 238 and 239) are formed. In some embodiments, one or more ports 230 are arranged to direct a fluid stream 210 along an oblique angle with respect to the central axis of the thrombus removal system. An operating pressure of the fluid delivery mechanism may be selected to approach a targeted fluid velocity for a fluid stream 210 that is delivered from a port 230. The targeted fluid velocity for a fluid stream 210 can be about 5 meters/second (m/s), about 8 m/s, about 10 m/s, about 12 m/s, or about 15 m/s. The targeted fluid velocity for fluid stream 210 can be any value within the range of aforementioned values. In some embodiments, at least two ports 230 are adapted to delivery respective fluid streams at different fluid velocities, for a given pressure of the fluid delivery mechanism. In some embodiments, at least two ports 230 are adapted to delivery respective fluid streams at the substantially the same fluid velocities, for a given pressure of the fluid delivery mechanism. In some embodiments, angular momentum is imparted to a thrombus by application of a) at least one fluid stream 210 that is directed at an oblique angle from a port 230, and/or b) at least two fluid streams 210 that have different fluid velocities. Advantageously, angular momentum produced in a thrombus may impart a (e.g., centrifugal) force that assists in fragmentation and removal of the thrombus. Advantageously, an increased cross-sectional area of the fluid lumen 145 reduces a required operating pressure of the fluid delivery mechanism to achieve a targeted fluid velocity of the fluid streams.

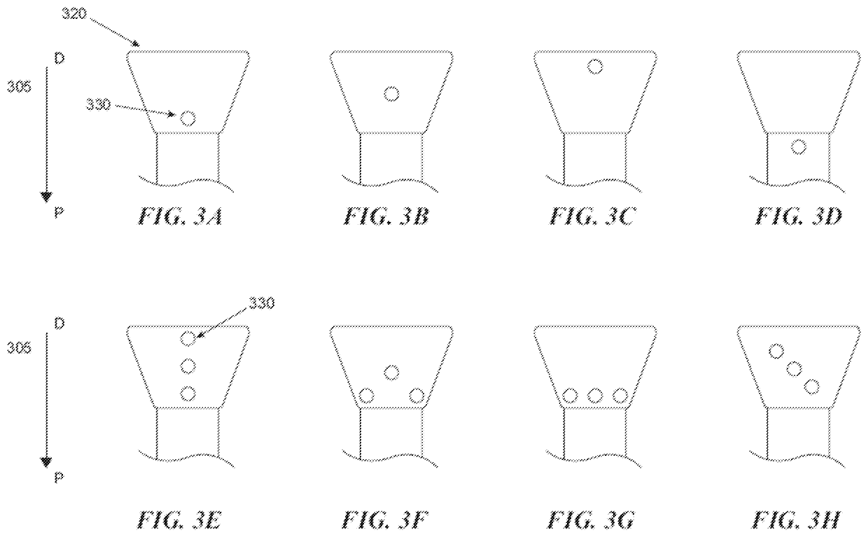

Referring to FIGS. 3A-3H, ports 330 can be arranged along various axial positions of the thrombus removal system. The thrombus removal system can include a flow axis 305 that is aligned with a general direction (e.g., distal-to-proximal) of flow for fluid that is aspirated therein. In some embodiments, a position of a port 330 comprises a) near a base of, b) in a middle portion of, c) in a distal portion of, or d) proximal to, a funnel portion 320 of the thrombus removal system. In some embodiments, at least two ports 330 are aligned along flow axis 305. In some embodiments, at least two ports 330 are arranged at a different axial positions along the flow axis 305. In some embodiments, at least two ports 330 are arranged (e.g., along a perimeter of the thrombus removal system) along a given axial position of the flow axis 305.

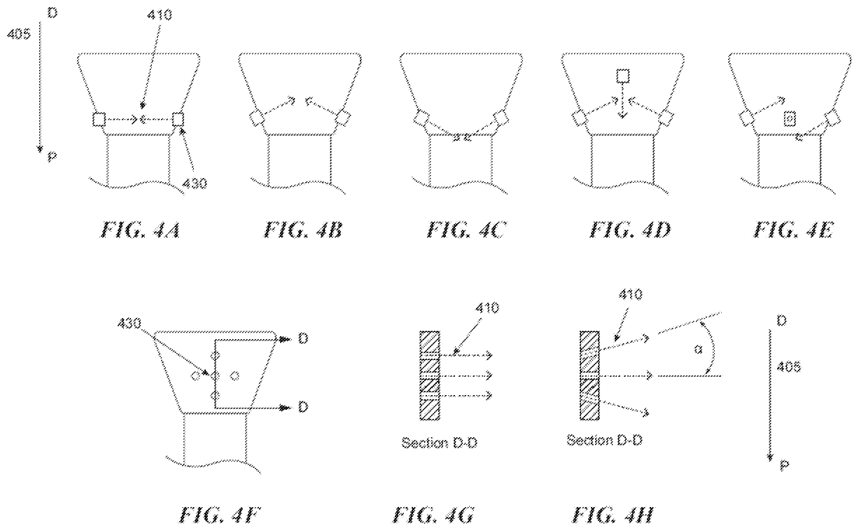

FIGS. 4A-4H depicts various configurations of fluid streams 410 that are directed from respective ports 430. A fluid stream 410 can be directed along a path that is substantially orthogonal, proximal, and/or distal to the flow axis 405 (which is like to flow axis 305). In some embodiments, at least two fluid streams are directed in different directions with respect to the flow axis 405. In some embodiments, at least two fluid streams are directed in a same direction (e.g., proximally) with respect to the flow axis 405. In some embodiments, at least a first fluid stream is directed orthogonally, at least a second fluid stream is directed proximally, and at least a third fluid stream is directed distally with respect to the flow axis 405. An angle a may characterize an angle a fluid stream 410 is directed with respect to an axis that is orthogonal to the flow axis 405 (e.g., as shown in section D-D of FIGS. 4G and 4H). An intersection region of fluid streams can be within an interior portion of the thrombus removal system, and/or exterior (e.g., distal) to the thrombus removal system. In some embodiments, a fluid stream that is directed by a port 430 in a nominal direction (e.g., distally) is deflected along an altered path (e.g., proximally) by (e.g., suction) pressure generated by the aspiration mechanism during operation.

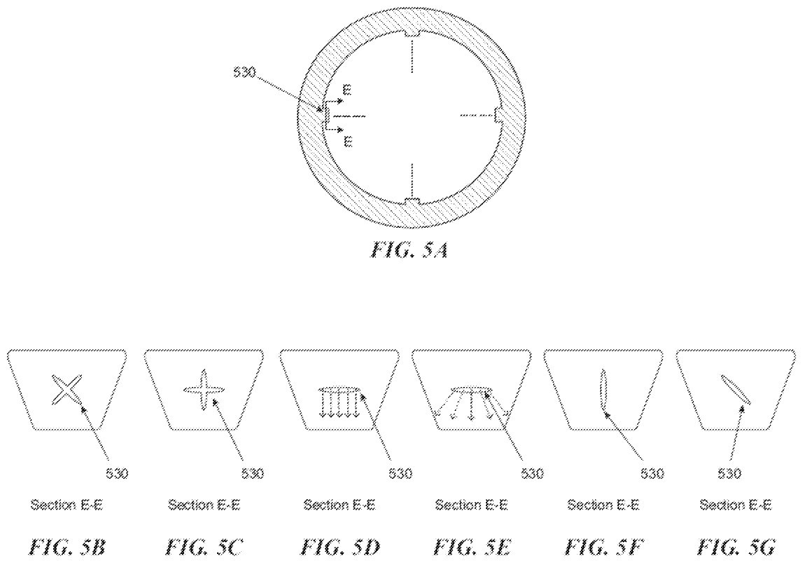

FIGS. 5A-5G illustrate a variety of exit aperture geometries with which ports 530 can be configured in accordance with embodiments of the present technology. Aperture geometries can comprise an oval, circular, cross (“x” shape), “t” shape, rectangle, or square shape. A fluid stream that is delivered from the port 530 can comprise substantially laminar flow (e.g., at the aperture), or a turbulent flow (e.g., that fans or outward).

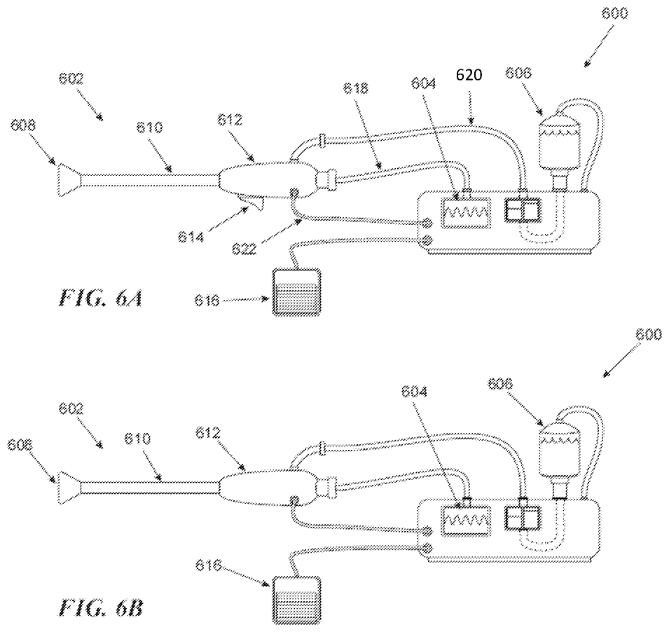

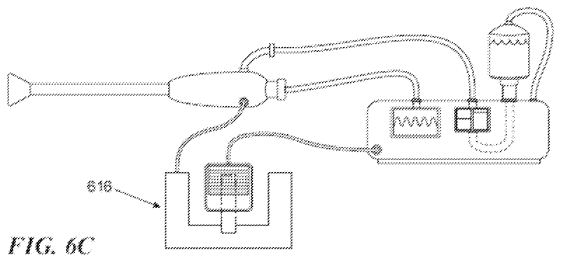

FIGS. 6A-6C illustrate various configurations of a thrombus removal system 600, including a thrombus removal device, 602, a vacuum source and cannister 604, and a fluid source 606. In some embodiments, the vacuum source and cannister and the fluid source are housed in a console unit that is detachably connected to the thrombus removal device. A fluid pump can be housed in the console, or alternatively, in the handle of the device. The console can include one or more CPUs, electronic controllers, or microcontrollers configured to control all functions of the system. The thrombus removal device 602 can include a funnel 608, a flexible shaft 610, a handle 612, and one or more controls 614 and 616. For example, in the embodiment shown in FIG. 6A, the device can include a finger switch or trigger 614 and a foot pedal or switch 616. These can be used to control aspiration and irrigation, respectively. Alternatively, as shown in the embodiment of FIG. 6B, the device can include only a foot switch 614, which can be used to control both functions, or in FIG. 6C, the device can include only an overpedal 616, also used to control both functions. It is also contemplated that an embodiment could include only a finger switch to control both aspiration and irrigation functions. As shown in FIG. 6A, the vacuum source can be coupled to the aspiration lumen of the device with a vacuum line 618. Any clots or other debris removed from a patient during therapy can be stored in the vacuum cannister 604. Similarly, the fluid source (e.g., a saline bag) can be coupled to the fluid lumens of the device with a fluid line 620.

Still referring to FIG. 6A, electronics line 622 can couple any electronics/sensors, etc. from the device to the console/controllers of the system. The system console including the CPUs/electronic controllers can be configured to monitor fluid and pressure levels and adjust them automatically or in real-time as needed. In some embodiments, the CPUs/electronic controllers are configured to control the vacuum and irrigation as well as electromechanically stop and start both system in response to sensor data, such as pressure data, flow data, etc.

As is described above, aspiration occurs down the central lumen of the device and is provided by a vacuum pump in the console. The vacuum pump can include a container that collects any thrombus or debris removed from the patient.

### Funnel Design

Various funnel shapes and design configurations are provided herein. Any of the funnels depicted in this disclosure can be used or included in thrombectomy catheters that include any of the other features described herein, including one or more jets and/or fluid streams configured to break up or macerate clots, and/or an aspiration lumen fluidly coupled to the funnel to aspirate clots out of the patient. In some embodiments, any of the funnels described herein can include jets or fluid streams within the funnel. In some embodiments, the jets or fluid streams can originate in the funnel itself. In other embodiments, the jets or fluid streams can be within the aspiration lumen, or at a distal end of the aspiration lumen. Additionally, any of the funnels disclosed or described herein can include mechanical manipulation or grasping elements, such as those described in PCT Application No. PCT/US2023/062002, filed on Feb. 2, 2023.

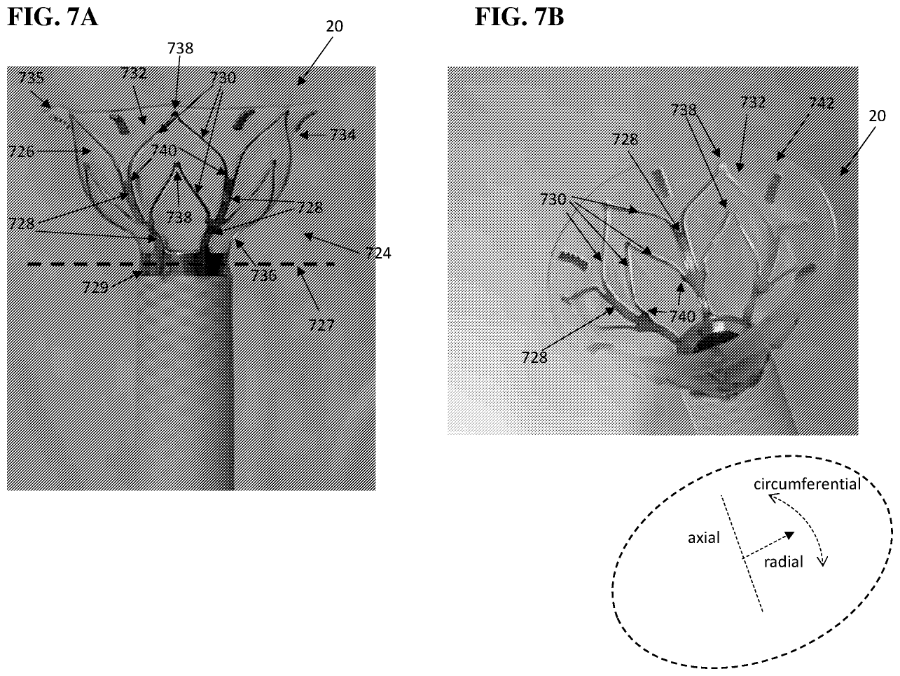

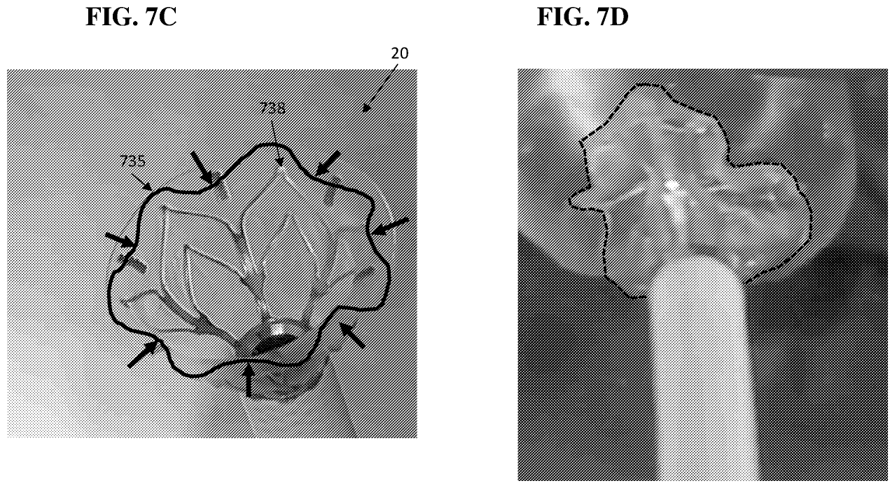

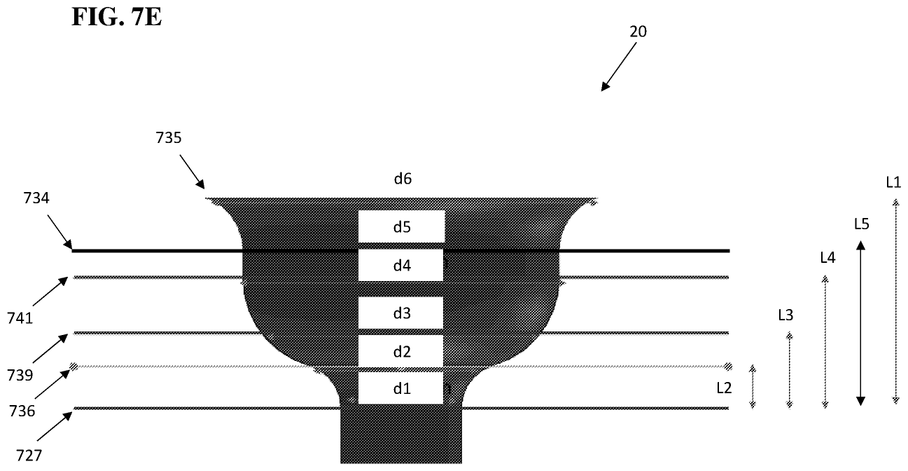

FIGS. 7A-7B show side and top views of a funnel 20 of a thrombectomy catheter which can include a shape that promotes clot capture during aspiration. The funnel 20 can include an expandable and collapsible frame 724 and a compliant membrane 726. The frame 724 is configured to assume a collapsed configuration (e.g., within a delivery sheath or catheter) to provide a reduced funnel diameter during delivery and navigation to a target thrombus location. When the delivery sheath or catheter is removed from the funnel, the frame 724 is configured to self-expand to assume a deployed configuration.

The frame 724 of the funnel can comprise a shape memory material, such as nitinol. The frame can comprise a complex structure that includes a plurality of axially extending spines 728 with adjacent spines 728 being connected with radially extending struts 730. The frame can further include a collar 729 configured to attach the funnel frame to a shaft of the thrombectomy catheter. In some embodiments, the spines, struts, and collar are integral to another (e.g., laser cut from a single sheet of material). In other embodiments, the spines, struts, and collar are separate components that are welded, glued, or attached together as is known in the art.

The catheter can further include fluid streams or jet streams to cut, break up, or macerate clot(s) pulled into one or more fluid planes or fluid regions (e.g., fluid plane 727). While not all embodiments of the thrombectomy catheter provided herein require the jets/fluid streams, the fluid plane 727 is shown as an exemplary reference for those embodiments that do include jets/fluid streams. The fluid plane 727 can also refer to a plane within which the jet or fluid ports sit. In embodiments without jets or fluid streams, reference 727 can refer to a narrow region within the funnel adjacent to or within the aspiration lumen. In FIG. 7A, the fluid plane 727 is shown generally in the collar 729 adjacent to or near the aspiration lumen. It should be understood that in other embodiments, the fluid region and/or jets can be in other locations. For example, the funnel itself can include jets, which could potentially move the fluid region into the funnel. In other embodiments, the fluid plane can be within the aspiration lumen. In some embodiments, there can be multiple fluid planes located through the funnel, collar, and/or aspiration lumen. Alternatively, the fluid plane need not be orthogonal to a longitudinal axis, but instead can be angled depending on if the jets are directed proximally, distally, or orthogonal to the longitudinal axis of the catheter.

As shown in FIGS. 7A-7B, each pair of adjacent spines 728 can be attached or connected to one or more sets (e.g., layers) of radially and/or circumferentially extending struts 730. In some embodiments, the spines 728 have a larger width or cross-section than the struts 730. Similarly, the spines 728 can be stiffer or more resistant to bending than the struts 730. In the embodiment of FIGS. 7A-7B, the frame includes two layers of radially extending struts 730 connecting adjacent spines 728. Each of the layers of radially extending struts can be displaced axially along the funnel from another. It can be seen how the combination of axially extending spines 728 and radially extending struts 730 form a petal 732 (resembling a chevron shape). In the illustrated embodiment, the frame includes two layers of petals, with six petals in each layer. It should be understood that other embodiments can include more or fewer layers of petals (e.g., up to 5 layers), and can include more or fewer petals per layer (e.g., up to 4 petals per layer, up to 8 petals per layer, up to 10 petals per layer) The petals in the first layer (e.g., on the proximal side of the funnel) can be smaller than the petals in the second layer (e.g., on the distal side of the funnel).

Still referring to FIGS. 7A-7B, the radially extending struts 730 can include one or more deflection points or hinges to reducing sheathing forces and facilitate more uniform and controllable collapse from the expanded configuration to the collapsed configuration. A first deflection point or hinge 738 is shown at the distal-most region of the struts (e.g., at the distal tip of each petal 732). As shown in FIG. 7A, each radially extending strut provides a radial connection between adjacent spines, but also extends slightly axially in the distal direction along the funnel. The distal most portion of each radially extending strut includes a hinge or deflection point 738. The frame structure does not include any radially or circumferentially extending struts without hinges or deflection points at the distal-most axial position of the radially extending strut. It is also noted that the hinges or deflection points 738 positioned centrally within each strut at the distal most axial position of the strut are also pointed or directed in the distal direction. The direction of the hinges 738 prevent the struts/hinges from getting caught up or stuck upon the sheathing catheter during re-sheathing. A second deflection point or hinge 740 is shown at each attachment or connection between the struts and the spines. The struts can be configured to bend or fold at the hinges 738 and 740 during sheathing or collapse of the funnel to reduce sheathing forces required for collapse of the funnel and also prevent or reduce flaring in the distal section of the funnel frame during sheathing. In some embodiments, the hinges 738 of the extending struts 730 can hinge in different planes. For example, the hinges 740 where the extending struts 730 connect or attach to the spines 728 can allow the struts 730 to bend in a radial plane or radial direction, and the hinges 738 at the distal-most portion of the struts 730 can allow the struts 730 to bend in a circumferential direction. This combination of axial struts, radially bending hinges and circumferentially bending hinges allows the funnel to maintain its structural integrity and preferred shape throughout the sheathing process while reducing sheathing forces (compared to frame structures without the described hinge arrangement).

Referring again to FIG. 7A, the funnel 20 can include a distal inversion in curvature and/or a distal flare 734 near the distal end of the funnel and a proximal inversion in curvature and/or a proximal flare 736 near the proximal end of the funnel. Collectively the distal flare 734 and the proximal flare 736 cause the funnel to assume a “bell” shape when in the expanded configuration. The funnel curvature and/or distal flare can reduce an incidence (e.g., rate) of clot becoming “hung” at the distal end of the funnel which prevents the clot from entering the funnel and/or causes pieces of clot to become lodged against an exterior of the funnel. For example, without the distal flare clots can wrap around or stretch over the edge of the funnel, causing a portion of the clot to be pulled down along an outer surface of the funnel when aspiration is activated. The distal flare 734 of the illustrated funnel provides a rolling surface to encourage the entirety of a clot to be directed into an interior of the funnel. The funnel curvature and/or proximal flare increases an interior diameter of the funnel in a proximal portion of the funnel near the jets/fluid streams and near the aspiration lumen. The proximal flare of the funnel is therefore configured to facilitate engagement and movement of clot(s) deep into the funnel to allow interaction between the clot and the jets/aspiration lumen. The proximal flare allows the funnel to collapse proximally when a clot is engaged with the funnel thereby bringing a portion of the clot to or across the fluid plane 727.

Additionally, since the distal end of the funnel and particularly the distal flare is so soft and compliant, a user can advance the thrombectomy device while the funnel is in the expanded configuration without undue concern of damaging the vessel walls. The distal most portion of the funnel is compliant and atraumatic enough that the device can be advanced in the expanded state. This enables simpler clot “hunting”, wherein minor adjustments to the funnel position relative to a clot can be made without requiring re-sheathing of the funnel.

As shown in FIGS. 7A-7B, proximal flare 736 can be formed as a result of a bend or inversion of curvature in the spines 728, and the distal flare 734 can be formed as a result of a bend or inversion of curvature in struts 730, particularly in the distal-most layer of struts 730. While the distal flare 734 is shaped as a result of the flaring or curvature of struts 730, it should be noted that the very distal-most section 742 of the funnel 20 does not include any rigid frame structures. This allows the distal-most section of the funnel to be extremely compliant relative to other portions of the funnel. This compliance in the distal flare of the funnel allows the distal end of the funnel to conform to clot(s) entering the funnel, which increases engagement with the clot(s) and limits the amount of blood aspirated into the catheter while the clot(s) are engaged.

FIGS. 7C-7D illustrate a deformation of the compliant distal end 735 that can occur when the funnel 20 engages with one or more clots. As described above, the distal end 735 of the funnel can comprise a compliant or flexible section of the funnel that does not contain any supporting frame structure in at least an axial portion of the distal end. When the funnel engages with a clot under aspiration, the compliant distal end is configured to bend and conform radially inwards to the clot, to seal or partially seal the funnel against the clot. This can result in portions of the distal end conforming, bending, or moving radially inwards as indicated by the arrows in FIG. 7C. Typically this bending or conforming occurs in the regions between hinges 738, where the frame structure does not advance as far distally in the axial direction. However, it should be understood that this bending or conforming can be non-uniform and can occur in sections even where hinges 738 are present. FIG. 7D is a photograph of a thrombectomy device engaged and sealed/partially sealed with a clot, which the compliant distal end of the funnel is shown bending or curving inwards to conform to the clot.

FIG. 7E shows another view of funnel 20, including the fluid plane 727, the proximal flare marked by an inversion of curvature of the funnel (e.g., at 736), the distal flare marked by yet another inversion of curvature of the funnel (e.g., at 734), and the distal end 735. Location 739 marks a position along the funnel just distal to the proximal flare, and location 741 marks a position along the funnel just proximal to the distal flare. The resulting cross-sectional shape of the funnel can comprise a hyperbolic parabola, with at least two inversions of curvature along its axial length, as shown. The funnel can optionally include a section between locations 741 and 734 where the diameter is constant (e.g., a relatively straight or uniform section of funnel). As described above, the funnel can have an axial length L1 between the distal end 735 and the fluid plane 727. The axial length L2 defines the distance between the fluid plane 727 and the proximal flare inversion 736, length L3 defines the distance between the fluid plane 727 and location 739, length L4 defines the distance between the fluid plane 727 and location 741, and length L5 defines the distance between the fluid plane 727 and the distal flare inversion 734. The proximal and distal flares, respectively, result in a funnel that has varying internal diameters. For example, the funnel can have a first diameter d1 at the fluid plane, which can be near or adjacent to where the funnel is coupled to the aspiration lumen of the thrombectomy catheter. In some embodiments, this diameter d1 is the narrowest section of the funnel. In some embodiments, the proximal-most diameter of the funnel is equal to a diameter of the aspiration lumen. Distal to this proximal-most section of the funnel, the diameter increases slightly until the proximal flare inversion 736, where the curvature of the funnel inverts and flares radially outwards. The diameter d2 of the funnel at the proximal flare can be larger than the diameter d1 at the fluid plane. As described above, increasing the diameter of the funnel so close to the aspiration lumen at the proximal flare facilitates advancing clots into and towards the jets and/or aspiration lumen of the device. In the section of the funnel between the proximal flare and the distal flare, the funnel can increase slightly in diameter, to a diameter d3 at location 739 and a diameter d4 at location 41. In some embodiments, the diameter of the funnel at location 739, just distal to the proximal flare, is similar or substantially equal to the diameter d5 of the funnel at the distal flare. Alternatively, the diameter d4 at location 741 is similar or substantially equal to the diameter d5 at the distal flare. The largest diameter of the funnel, d6, is shown at the distal end of the funnel. The distal flare 734 provides another inversion of the curvature of the funnel resulting in the distal tip or end 735 of the funnel pointing radially outwards, as shown and described herein.

In terms of the axial length of the funnel, the length L2 between the fluid plane and the proximal flare can range between 15-25 percent of the length of the funnel L1. The length L3 between the fluid plane and location 739 (just distal to the proximal flare) can range between 30-40 percent of the length of the funnel L1. The length L5 between the fluid plane and the distal flare can range between 60-80 percent of the funnel L1.

Still referring to FIG. 7E, in some embodiments, a diameter d1 of the funnel at the distal flare 734 is approximately equal to a diameter of the funnel at location 739 just distal to the proximal flare 736. In other embodiments, the diameter of the funnel at location 739 is within 10%, or optionally, within 20% of the diameter of the funnel at the distal flare. In one specific example, the diameter d1 of the funnel at the fluid plane can be in the range of 2-4 mm, while the diameter d6 at the distal tip 735 of the funnel can be in the range of 10-14 mm. Similarly, the diameter d5 at the distal flare can be in the range of 7-9 mm, and the diameter d2 at the proximal flare 736 can be in the range of 4-8 mm. Furthermore, the proximal flare can start approximately 0.5 to 1.5 mm away from the fluid plane or aspiration lumen of the device. The resulting funnel therefore includes a very wide diameter section starting at the proximal flare that is placed very close relative to the aspiration lumen, advantageously bringing clots, including large clots, into the cutting plane of the jets of the thrombectomy catheter.

As described above, the funnel can include a compliant material 726 disposed on or over the frame 724. The compliant material can comprise elastomeric materials such as a polycarbonate-based urethane such as chronoflex or other similar material. In some embodiments, the compliant material includes one or more layers. For example, the funnel can include a first layer of compliant material on an interior of the funnel and a second layer of a compliant material on an exterior of the funnel. Alternatively, when spraying a compliant material on the frame, the funnel may include more layers of the compliant material in the “windows” between the frame structure than the amount of layers adhered to the frame structure itself. In some embodiments, the two or more layers can “sandwich” the funnel frame 724. In another embodiment, an interior layer of compliant material can be used inside the frame 724, and the exterior layer can be spray coated on the outside of the frame to attach the interior layer to the frame. In some embodiments, the interior compliant material layer can comprise a different material than an exterior compliant material. For example, the interior compliant material layer can comprise a low-tack or lubricious material configured to encourage the clot to slip or slide within the funnel instead of sticking or adhering to the funnel. This low-tack or lubricious material can encourage the clot to move within the funnel towards the jets and/or aspiration lumen. In some embodiments, a lubricious material like polytetrafluoroethylene (PTFE) or expanded polytetrafluoroethylene (ePTFE) can be used on an interior surface of the funnel.

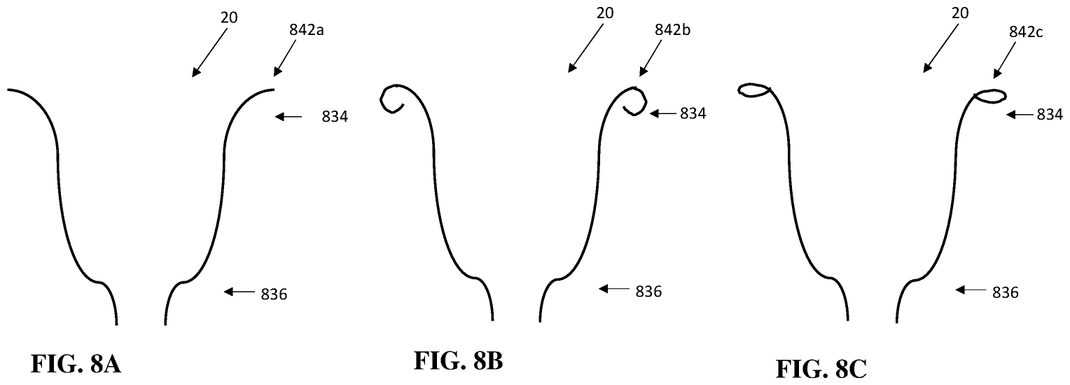

FIGS. 8A-8C illustrate cross-sectional views of a funnel 20 with various distal funnel edge variations. For example, the distal flare 834 shown in the embodiment of FIG. 8A can be similar to the distal flare 734 of described above in FIGS. 7A-7B. In this example, the distal flare comprises an inversion of curvature in the funnel with the distal edge 842a of the funnel pointing radially outwards (e.g., towards a vessel wall when the funnel is expanded). The embodiment of FIG. 8A further shows section 835 of the funnel which includes the axial section of funnel extending between the proximal flare 836 and the distal flare 834. It can be seen from FIG. 8A that while the diameter of the funnel increases between the proximal flare and the distal flare, this rate of increase is smaller than the rate of diameter increase through the proximal and distal flares.

In contrast, the embodiment of FIG. 8B shows a distal flare 834 in which a distal edge 842b of the funnel wraps or curves around and inward back towards the distal flare (e.g., resembling a ram's horn). It is noted that in this embodiment while the distal edge curves or folds back inwards towards the funnel, the distal edge itself is not sealed against the funnel but instead forms an open lip or ring around the distal tip of the funnel.

In the embodiment of FIG. 8C, the distal edge 842c of the funnel curves or folds back and is sealed or bonded against the funnel to form a beaded or folded edge. All variations of the distal flare and distal edge of the funnel are designed and configured to prevent clots from catching or hanging up on the distal edge of the funnel and to encourage the clot(s) into the interior of the funnel.

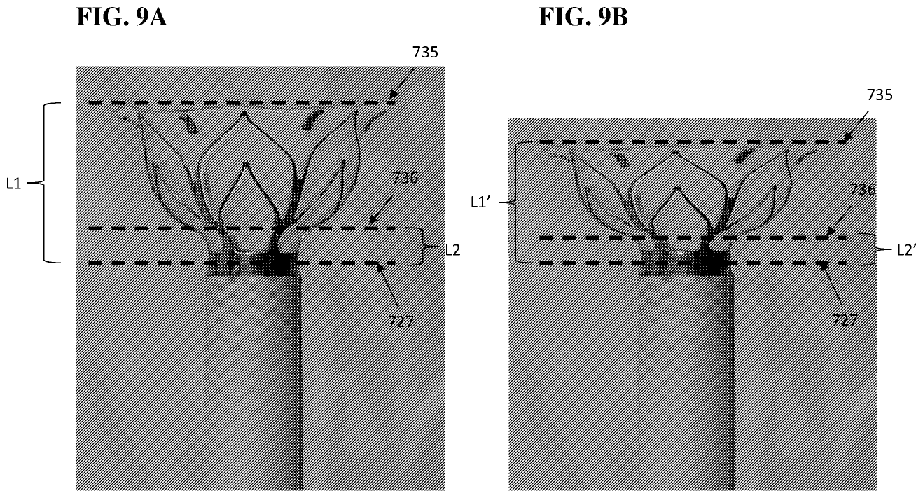

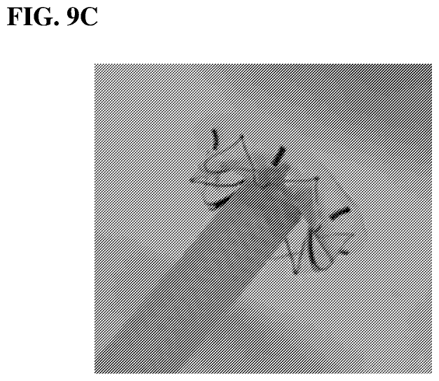

Alternatively or additionally, the funnel curvature and/or distal flare can promote a shape change in the funnel under aspiration or during advancement of the funnel within a vessel. FIGS. 9A-9B illustrate two configurations of the funnel to illustrate how the funnel responds to dead-heading or advancing the funnel directly into a rigid structure such as a vessel wall. In FIG. 9A, the funnel is shown in an expanded or deployed configuration prior to engagement with a wall. Length L1 represents the axial distance between the fluid plane 727 and the distal end 735 of the funnel. Length L2 represents the axial distance between the fluid plane 727 and the proximal flare 736. FIG. 9B shows the funnel in the expanded or deployed configuration after dead-heading or being directed forcefully into a wall or other rigid structure. The distal end 735 of the funnel has the compliance and flexibility to bend and collapse, resulting in the funnel foreshortening in the axial direction. In FIG. 9B, the distance between the distal end 735 and the fluid plane foreshortens to a length L1' and the distance between the proximal flare 736 and the fluid plane 727 foreshortens to a length L2′. It is noted that the diameter of the funnel at the proximal flare 736 may also change (e.g., increase) when this foreshortening occurs, as the funnel frame in this section bows (e.g., outward) to account for the axial foreshortening. Further advancement of the catheter can continue to change the shape the shape of the funnel and result in an eversion, for example, of the distal portion of the funnel, as shown in FIG. 9C. Even in an everted configuration as shown in FIG. 9C, the combination of jets and aspiration can still remove clots in this configuration. However, with the funnel not being able to form an ideal seal or partial seal with a clot in this configuration, the resulting treatment can result in a clot “bouncing” against the distal end of the device as the clot engages with the aspiration lumen and is partially cut or macerated with the jets.

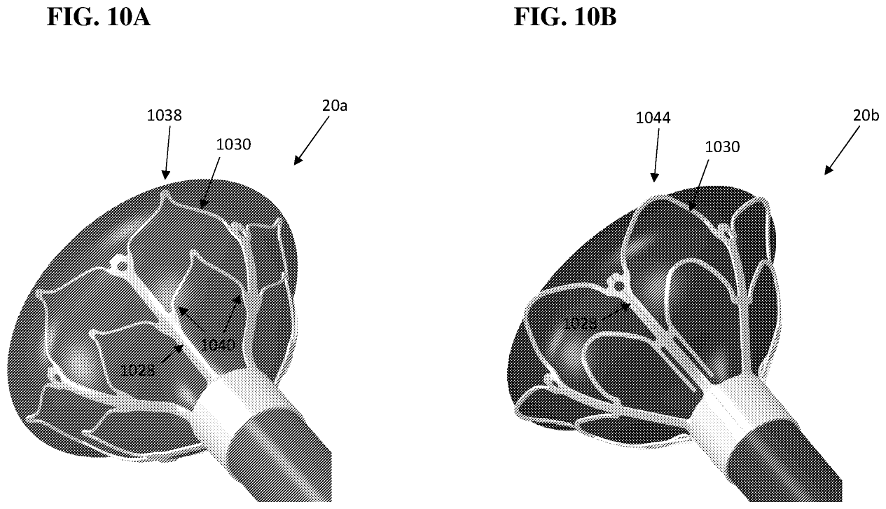

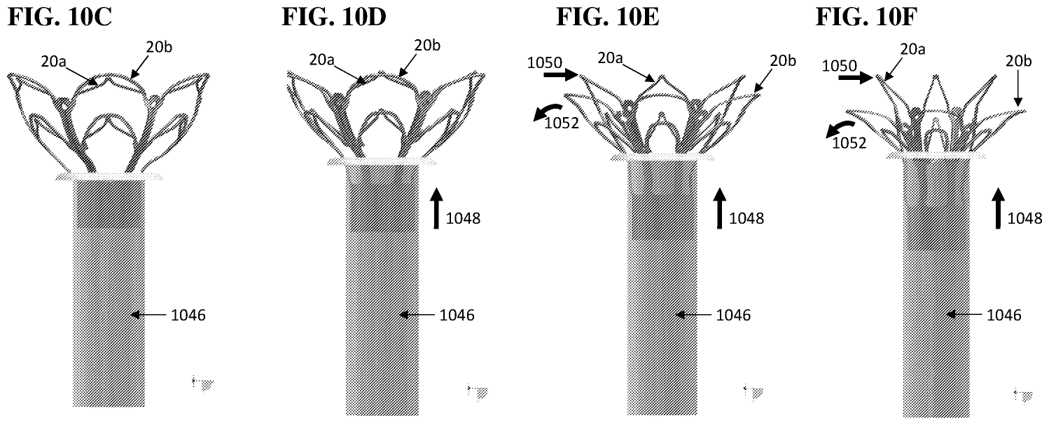

FIGS. 10A-10B illustrate additional views of a funnel 20a/20b including a funnel frame. The funnel 20a of FIG. 10A can include the funnel frame of FIGS. 7A-7B, in which the radially extending struts 1030 include a deflection point or hinge 1038 along a length of each radially extending strut (e.g., at the distal most portion of each strut). Typically, this hinge or deflection point will be in a central portion of the strut. The radially extending struts 1030 can also include deflection points or hinges 1040 where the struts attach or connect to the axially extending spines 1028, as previously described. In contrast, the funnel 20b of FIG. 10B includes struts 1030 that do not have hinges or deflection points, but instead comprise a smooth curve 1044 along the span between adjacent spines 1020. As a result, the funnel 20b is more resistant to sheathing, requiring additional sheathing forces to collapse the funnel. Furthermore, the embodiment of FIG. 10B is prone to radial flaring during sheathing, particularly at the distal portion of the funnel.

FIGS. 10C-10F illustrate sheathing of funnels 20a and 20b (overlaid upon another for easy of illustration) with a delivery sheath or catheter 1046. It should be understood that funnels 20a and 20b are illustrated together to show the difference in sheathing between the funnels. FIG. 10C shows both funnels 20a and 20b in a fully expanded or deployed configuration, with the delivery sheath or catheter 1046 pulled proximally clear of the funnel frame. In FIG. 10D, the delivery sheath or catheter 1046 is advanced distally over a proximal portion of the funnel frame in direction 1048 to contact the frame and/or funnel membrane (not shown) and start the sheathing process. In FIG. 10E, the delivery sheath or catheter 1046 is further advanced in direction 1048 to continue sheathing funnels 20a and 20b. Here, the difference in sheathing between funnel 20a and 20b becomes more apparent. Since the radially extending struts of funnel 20a include hinges or deflection points as previously described, sheathing of the funnel causes the struts to collapse or fold along the hinges or deflection points, resulting in the distal tip of funnel 20a moving inwards in direction 1050. In contrast, the distal tip of funnel 20b begins to flare outwards while also moving in the proximal direction as indicated by arrow 1052, since the struts do not have feature configured to promote bending or hinging. While the funnel 20b has an increased axial profile during sheathing compared to funnel 20b, the funnel 20b also requires greater sheathing forces from delivery sheath or catheter 1046. FIG. 10F shows further advancement of delivery sheath or catheter 1046 in direction 1048. In this figure, the radial flaring of funnel 20b is very pronounced compared to the smooth inward collapse of funnel 20a.

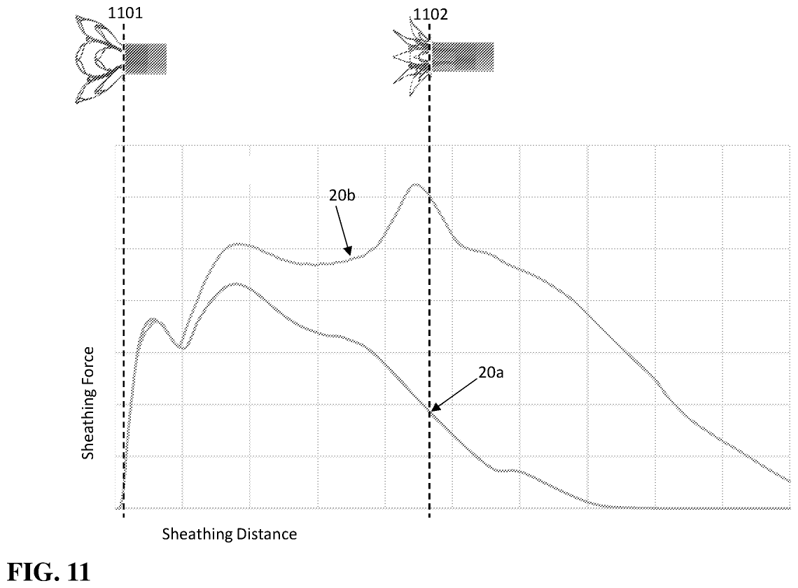

FIG. 11 is a chart showing the sheathing force required for funnel 20b compared to the sheathing force required for funnel 20a. The top of the chart shows the various degrees of funnel sheathing as described above in FIGS. 10C and 10F. Line 1101 corresponds to the sheathing forces of funnels 20a and 20b as illustrated in FIG. 10C and line 1102 corresponds to the sheathing forces of funnels 20a and 20b as illustrated in FIG. 10F. As shown, the required sheathing forces for funnel 20a are significantly lower than those required for funnel 20b, particularly early in sheathing. Funnel 20a requires approximately 20% to 80% lower sheathing forces at depending on the level of sheathing. The reduced sheathing forces are a direct result of the deflection points or hinges in the struts, both within each radially extending strut and also at the attachment or connection point between the axially extending spines and the radially extending struts.

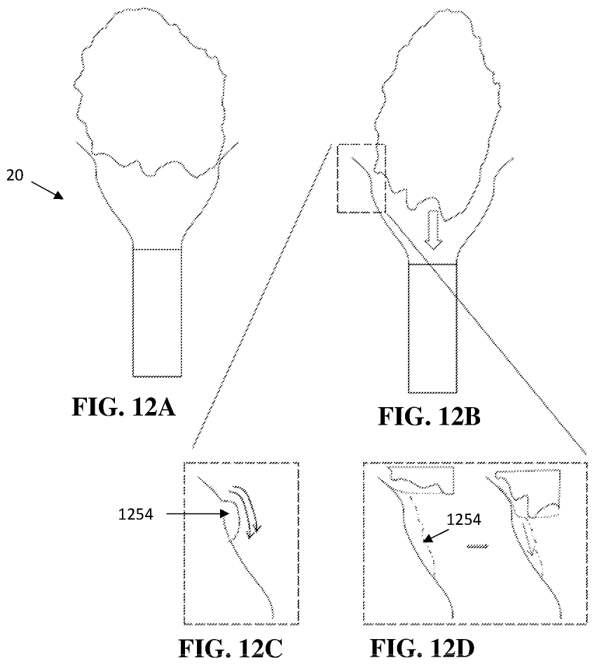

Referring to FIGS. 12A-12D, some embodiments of the funnel 20 can include aspiration elements adapted to enhance aspiration forces within the thrombectomy catheter and/or funnel. FIG. 12A depicts a “hard” clot that can become lodged at the distal region of the thrombectomy catheter (e.g., funnel). FIG. 12B depicts a thrombectomy catheter having one or more aspiration elements 1254 disposed on or within the funnel, which are adapted to enhance clot capture. As shown in FIG. 12C, an aspiration element 1254 can include a protrusion (e.g., a rib) that is positioned near a distal end of the funnel. The protrusion can be formed into the funnel (e.g., into the compliant material) or separately bonded or adhered to the interior of the funnel. The protrusion can be present in at least a portion (e.g., an entirety) of the (e.g., inner) perimeter of the funnel. The aspiration element can locally decrease pressure within the funnel to accelerate flow. As shown in FIG. 12D, an aspiration element can include a compliant member (e.g., skirt). The compliant skirt can (e.g., passively) adapt its shape when a clot is adjacent thereto. The change in shape can alter an area and/or volume within which the applied aspiration, enhancing the aspiration forces applied to the clot.

While the embodiments herein have been described as being intended to remove thrombi from a patient's vasculature, other applications of this technology are provided. For example, the devices described herein can be used for breaking up and removing hardened stool from the digestive tract of a patient, such as from the intestines or colon of a patient. In one embodiment, the device can be inserted into a colon or intestine of the patient (such as through the anus) and advanced to the site of hardened stool. Next, the aspiration system can be activated to engage the hardened stool with an engagement member (e.g., funnel) of the device. Finally, the jets or irrigation can be activated to break off pieces of the hardened stool and aspirate them into the system. Any of the techniques described above with respect to controlling the system or removing clots can be applied to the removal of hardened stool.

As one of skill in the art will appreciate from the disclosure herein, various components of the thrombus removal systems described above can be omitted without deviating from the scope of the present technology. As discussed previously, for example, the present technology can be used and/or modified to remove other types of emboli that may occlude a blood vessel, such as fat, tissue, or a foreign substance. Further, although some embodiments herein are described in the context of thrombus removal from a pulmonary artery, the disclosed technology may be applied to removal of thrombi and/or emboli from other portions of the vasculature (e.g., in neurovascular, coronary, or peripheral applications). Likewise, additional components not explicitly described above may be added to the thrombus removal systems without deviating from the scope of the present technology. Accordingly, the systems described herein are not limited to those configurations expressly identified, but rather encompasses variations and alterations of the described systems.

### Conclusion

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A thrombus removal device, comprising:
an elongate catheter having an aspiration lumen configured to be coupled to an aspiration source, and defining a longitudinal axis;
an expandable funnel coupled to the aspiration lumen and the elongate catheter, the expandable funnel including a proximal flared portion near the aspiration lumen and a distal flared portion near a distal end of the expandable funnel; and
one or more fluid ports disposed near or within the expandable funnel defining a fluid region.

2. The device of claim 1, wherein a first diameter of the expandable funnel at the proximal flared portion is within 10% of a second diameter of the expandable funnel at the distal flared portion.

3. The device of claim 1, wherein a first diameter of the expandable funnel at the proximal flared portion is within 20% of a second diameter of the expandable funnel at the distal flared portion.

4. The device of claim 1, wherein the proximal flared portion is configured to facilitate advancement of clots into the fluid region.

5. The device of claim 1, wherein the distal end of the expandable funnel is directed radially outwards.

6. The device of claim 1, wherein the distal end of the expandable funnel curves proximally back towards the distal flare.

7. The device of claim 6, wherein the distal end is not bonded to the distal flare.

8. The device of claim 6, wherein the distal end is bonded to the distal flare.

9. The device of claim 1, wherein the expandable funnel further comprises a funnel frame.

10. The device of claim 9, wherein the funnel frame comprises a shape memory material.

11. The device of claim 1, wherein the funnel frame comprises a plurality of axially extending spines and one or more layers of radially and/or circumferentially extending struts between adjacent spines.

12. The device of claim 1, wherein the proximal flare is formed as a result of a bend in each of the plurality of axially extending spines.

13. The device of claim 1, wherein the distal flare is formed as a result of a bend in each of the plurality of radially and/or circumferentially extending struts.

14. The device of claim 11, further comprising at least one hinge in each of the plurality of radially and/or circumferentially extending struts.

15. The device of claim 14, wherein a first hinge is disposed at a distal most portion of each of the plurality of radially and/or circumferentially extending struts.

16. The device of claim 15, wherein a second hinge is disposed at a junction between one of the plurality of radially extending struts and one of the plurality of axially extending spines.

17. The device of claim 14, wherein the at least one hinge is configured to reducing a sheathing force of the expandable funnel.

18. The device of claim 14, wherein the at least one hinge is integral to the plurality of radially and/or circumferentially extending struts.

19. The device of claim 11, wherein the plurality of axially extending spines are stiffer than the plurality of radially and/or circumferentially extending struts.

20. The device of claim 11, wherein the plurality of axially extending spines have a thickness greater than that of the plurality of radially and/or circumferentially extending struts.

21. The device of claim 11, wherein the funnel frame comprises two layers of radially extending struts.

22. The device of claim 11, wherein the plurality of axially extending spines and radially extending struts collectively form a plurality of petals in each of the one or more layers.

23. The device of claim 22, wherein petals in a proximal layer of the funnel frame are smaller than petals in a distal layer of the funnel frame.

24. The device of claim 22, further comprising at least six petals in each of the one or more layers.

25. The device of claim 9, further comprising a compliant material attached to the funnel frame.

26. The device of claim 24, wherein a distal-most portion of the expandable funnel comprises only compliant material with no funnel frame.

27. A catheter device, comprising:
an elongate catheter having lumen therethrough;
an expandable funnel coupled to the elongate catheter at a distal end thereof, the expandable funnel including a funnel frame comprising:
a plurality of axially extending spines;
one or more sets of radially and/or circumferentially extending struts between adjacent spines; and
one or more hinges disposed on or in the plurality of radially and/or circumferentially extending struts, the one or more hinges adapted to flex circumferentially and/or radially to modify a geometry of the expandable funnel.

28.-77. (canceled)
